(19) United States
(12) Patent Application Publication
(10) Pub. No.: US 2024/0091170 A1
(43) Pub. Date: Mar. 21, 2024

(54) Catechol Nanoparticle, Catechol Protein Nanoparticle, and Preparation Method and Use Thereof

(71) Applicant: Shihezi University, Shihezi City (CN)

(72) Inventors: Bo HAN, Shihezi City (CN); Jingmin Fan, Shihezi City (CN); Hang Yu, Shihezi City (CN); Rui Xue, Shihezi City (CN); Jiawei Guan, Shihezi City (CN); Yu Xu, Shihezi City (CN); Linyun He, Shihezi City (CN); Ji Liu, Shihezi City (CN); Chengyu Jiang, Shihezi City (CN); Xin Lu, Shihezi City (CN); Xiangze Kong, Shihezi City (CN); Wei Yu, Shihezi City (CN); Wen Chen, Shihezi City (CN)

(73) Assignee: Shihezi University, Shihezi City (CN)

(21) Appl. No.: 18/228,761

(22) Filed: Aug. 1, 2023

(30) Foreign Application Priority Data
Aug. 4, 2022 (CN) 202210932024.5

(51) Int. Cl. A61K 31/05; A61K 9/14; A61K 9/51; A61K 36/22; A61K 36/49; A61K 36/739; A61K 38/17; A61K 38/36; A61K 38/38; A61K 38/41; A61K 38/44; A61K 38/47; A61K 38/48; A61K 39/395

(52) U.S. Cl. CPC A61K 31/05; A61K 9/14; A61K 9/5192; A61K 36/22; A61K 36/49; A61K 36/739; A61K 38/1722; A61K 38/363; A61K 38/385; A61K 38/415; A61K 38/44; A61K 38/443; A61K 38/47; A61K 38/488; A61K 39/39516

(57) ABSTRACT

Provided are catechol nanoparticles, catechol protein nanoparticles, and a preparation method and use thereof. The method includes: adding a tannin compound-containing natural herb medicine into water to obtain a mixture, and subjecting the mixture to heating reflux extraction to obtain a herb medicine extract and subjecting the herb medicine extract to fractionation to obtain the catechol nanoparticles.

### CROSS REFERENCE TO RELATED APPLICATION

This patent application claims the benefit and priority of Chinese Patent Application No. CN202210932024.5, entitled “CATECHOL NANOPARTICLE, CATECHOL PROTEIN NANOPARTICLE, AND PREPARATION METHOD AND USE THEREOF” filed with the China National Intellectual Property Administration (CNIPA) on Aug. 4, 2022, which is incorporated herein by reference in its entirety.

### TECHNICAL FIELD

The present disclosure relates to the technical field of medicines, in particular to catechol nanoparticles, catechol protein nanoparticles, and a preparation method and use thereof.

### BACKGROUND

Chinese herbal medicine nanoparticles are nanoscale biofunctional materials made of Chinese herbal medicines using nanotechnology to enhance and improve the therapeutic effects of traditional Chinese medicine in various diseases. At present, methods for preparing Chinese herbal medicine nanoparticles include:
(1) Self-nanocrystallization of a herb medicine: a drug, a grinding medium, water, and a corresponding stabilizer are put into a special medium grinder, obtaining a mixture. The mixture is subjected to high-speed shearing through a grinding rod to grind, obtaining a nanoscale drug. The methods for grinding include mechanical crushing and ball milling. This process of self-nanocrystallization has defects and shortcomings: the application is limited, and the morphology of nano-drugs obtained is generally irregular. Moreover, for some herb medicines with high viscosity, the mechanical crushing and ball milling are not applicable.
(2) The herb medicine is loaded into a specific nanocarrier, such as solid liposome, nanoemulsion, or nanomicelle, by methods, such as self-assembly, micro-emulsion method, and thin-film rehydration method. The process has defects and shortcomings: the preparation process is complex, relies on modern scientific precision instruments, and is difficult to achieve mass industrial production.

### SUMMARY

An object of the present disclosure is to provide catechol nanoparticles, catechol protein nanoparticles, and a preparation method and use thereof. The method may easily and quickly prepare spherical nanoparticles with regular shapes from a herb medicine extract, thereby realizing industrial production.

To achieve the above object, the present disclosure provides the following technical solutions:

The present disclosure provides a method for preparing catechol nanoparticles, including:
adding a tannin compound-containing natural herb medicine into water to obtain a mixture, and subjecting the mixture to heating reflux extraction to obtain a herb medicine extract; and
subjecting the herb medicine extract to fractionation to obtain the catechol nanoparticles.

In some embodiments, the tannin compound-containing natural herb medicine includes one drug selected from the group consisting of Quercus infectoria Oliv. (Turkish galls), Rhus chinensis Mill., and Sanguisorba officinalis L.; a ratio of a mass of the tannin compound-containing natural herb medicine to a volume of water is 1 g: 8 mL; and the heating reflux extraction is conducted by atmospheric reflux extraction or vacuum reflux extraction, where the atmospheric reflux extraction is conducted at 100° C. for 2 h, and the vacuum reflux extraction is conducted at 50° C. for 2 h.

In some embodiments, the fractionation is conducted by a process including the following steps: subjecting the herb medicine extract to first centrifugal separation to obtain a supernatant and the catechol nanoparticles; and the first centrifugal separation is conducted at a centrifugal force of 6,577 g for 10 min.

In some embodiments, the fractionation further includes: subjecting the supernatant to a second centrifugal separation to obtain the catechol nanoparticles; and the second centrifugal separation is conducted at a centrifugal force of 9,500 g for 10 min.

The present disclosure further provides catechol nanoparticles prepared by the method described in the above technical solutions, where the catechol nanoparticles are formed by self-assembly of a catechol and a protein, and have an average particle size of 413.89 nm±202.95 nm or 230.34 nm±59.48 nm.

The present disclosure further provides a method for preparing catechol protein nanoparticles, including:
preparing a herb medicine extract by the method described in the above technical solutions; and
subjecting the herb medicine extract to first centrifugal separation to obtain a first supernatant, subjecting the first supernatant to second centrifugal separation to obtain a second supernatant, mixing the second supernatant, water, and a protein to obtain a mixture, subjecting the mixture to self-assembly under heating, and conducting separation to obtain the catechol protein nanoparticles.

In some embodiments, the protein includes one protein selected from the group consisting of bovine serum albumin (BSA), lysozyme (LYZ), cytochrome C (CYC), β-lactoglobulin (bLG), pepsin, β-galactosidase (β-gal), hemoglobin (Hgb), fibrinogen (FGN), immunoglobulin G (IgG), horseradish peroxidase (HRP), and glucose oxidase (GOX); and a mass ratio of the protein to the second supernatant is in a range of (10-300): 600.

In some embodiments, the heating is conducted at 100° C.; the self-assembly is conducted for 2 h; the separation is conducted by centrifugal separation; and the centrifugal separation is conducted at a centrifugal force of 5,000 g for 10 min.

The present disclosure further provides catechol protein nanoparticles prepared by the method described in the above technical solutions.

The present disclosure further provides use of the catechol nanoparticles described in the above technical solutions or the catechol protein nanoparticles described in the above technical solutions as a drug carrier.

The present disclosure provides a method for preparing catechol nanoparticles. In the present disclosure, the tannin compound-containing natural herb medicine is used as a raw material, and catechol compounds and proteins are subjected to self-assembly by heating reflux; and natural nanoparticles-catechol nanoparticles with a regular shape may be prepared by simple fractionation. The formation mechanism of the catechol nanoparticles is the self-assembly of small-molecular-weight catechol compounds (with a mass-to-charge ratio of less than 635) and trace proteins in the Quercus infectoria Oliv. to form nanoparticles. The nanoparticles prepared by the method have pH responsiveness and free radical scavenging capacity, while a part of the protein may retain its biological activity. Therefore, the nanoparticles may be used as a drug carrier for drug delivery and therapy.

In the present disclosure, the herb medicine extract is extracted by heating reflux. On the one hand, the heating is conducive to high-speed movement between the molecules, and the solution is thermally stirred to make the molecules enter the self-assembled aggregate in an appropriate direction to obtain a more ordered and organized final state. On the other hand, the heating provides a driving force for the structural changes in proteins. Protein molecules contain hydrophobic groups inside. When the protein structure changes, the hydrophobic groups are exposed, and non-polar molecules or non-polar groups approach each other and accumulate in an aqueous solution. The process of heating and boiling may cause structural changes in the protein, that is, the partial unfolding of the tertiary structure and the conformational changes of the secondary structure. In this way, specific regions (such as hydrophobic sites or free-SH groups) are easily exposed, and proteins form aggregates through hydrophobic interactions, thereby obtaining natural spherical nanoparticles with regular shapes.

Furthermore, in the present disclosure, natural spherical nanoparticles with different particle size distributions, uniform particle size, and regular shape may be obtained easily and quickly by using the herb medicine extract through differential centrifugation.

The present disclosure further provides a method for preparing catechol protein nanoparticles. After different proteins are additionally added to the herb medicine extract, a large number of natural nanoparticles may be obtained through self-assembly, such that a large number of natural nanoparticles can be industrially produced. In the present disclosure, the preparation method has environmental friendliness, a simple preparation process, a short time, and a high yield, and may realize mass industrial production.

### BRIEF DESCRIPTION OF THE DRAWINGS

FIG. 1 is a schematic diagram showing the flow process and mechanism for preparation of the Quercus infectoria Oliv. catechol nanoparticles according to an embodiment of the present disclosure and the performance of the Quercus infectoria Oliv. catechol nanoparticles.

FIG. 2A to FIG. 2G show characterization results of the Quercus infectoria Oliv. catechol nanoparticles prepared in Example 1; where FIG. 2A represents SEM (Scanning Electron Microscope) and TEM (Transmission Electron Microscope) images of TG-LP nanoparticles; FIG. 2B represents SEM and TEM images of TG-HP NPs, and the inset represents a representative appearance of the nanoparticles; FIG. 2C represents a particle size distribution of TG-LP NPs; FIG. 2D represents a particle size distribution of TG-HP NPs; FIG. 2E represents a UV-visible absorption spectrum of TGE, TG-LP NPs, and TG-HP NPs at the same concentration; FIG. 2F represents a Fourier transform infrared spectrum of TGE, TG-LP, and TG-HP NPs; and FIG. 2G represents a Zeta potential diagram of Quercus infectoria Oliv. catechol nanoparticles TG-LP NPs and TG-HP NPs.

FIG. 3A to FIG. 3D show the performance test results of the Quercus infectoria Oliv. catechol nanoparticles prepared in Example 1; where FIG. 3A and FIG. 3B represent the results of the antioxidant activity and the experimental results of the nanoparticles in scavenging free radicals; FIG. 3C represents the change in intensity of the Tyndall effect and the change in mass and polyphenol content of the nanoparticles with the pH value after co-incubating and shaking the nanoparticles with buffer solutions of different pH values for 30 min; and FIG. 3D represents an SEM image of the nanoparticles after co-incubating and shaking the nanoparticles with buffer solutions of different pH values for 30 min.

FIG. 4 shows a standard curve of the gallic acid.

FIG. 5 is a result map showing the chemical composition of TGE, TG-LP NPs, and TG-HP NPs measured by LC-MS.

FIG. 6 is a thermal map showing the content variation of main components in different nanoparticles.

FIG. 7 shows the chemical structural formulas of 9 kinds of catechol series in Quercus infectoria Oliv.

FIG. 8 shows an energy dispersive X-ray (EDX) analysis diagram of the TG-LP NPs, scale bar: 200 nm.

FIG. 9 shows a schematic diagram of the ninhydrin reaction.

FIG. 10 shows a solution picture after adding a ninhydrin chromogenic solution in the positive control group (BSA) and the sample groups (Quercus infectoria Oliv. extract TGE, Quercus infectoria Oliv. catechol nanoparticles TG-LP NPs and TG-HP NPs).

FIG. 11A represents a schematic diagram of the self-assembly of TG-HS and protein; FIG. 11B represents an SEM image of TG-HS (600.00 mg) and TG-BSA NPs formed by feeding different amounts of BSA (b1 to b6: 10.00 mg, 30.00 mg, 50.00 mg, 100.00 mg, 200.00 mg, 300.00 mg); b7, b8: TEM images of TG-BSA NPs with BSA feeding amount of 30.00 mg; FIG. 11C represents an EDX analysis diagram of TG-BSA NPs; and FIG. 11D represents an SEM image of TG-BSA NPs incubated with different pH buffer solutions for 30 min; inset: photos of TG-BSA NPs added to different pH buffer solutions (3.0, 7.0, 9.0, and 11.0).

FIG. 12A represents a model protein map showing different molecular weights, fat index, and isoelectric point; (x-axis: isoelectric point; y-axis: molecular weight; z-axis: aliphatic index); and FIG. 12B represents an SEM image showing different extraction states during the self-assembly of Quercus infectoria Oliv. extract TG-HS and 10 kinds of proteins to form nanoparticles.

FIG. 13 shows an X-Gal staining result map of A549 cells treated with TG-β-Gal NPs and TG-β-gal NPs (A) for 24 h, where a control group is treated with free β-Gal.

### DETAILED DESCRIPTION OF THE EMBODIMENTS

The present disclosure provides a method for preparing catechol nanoparticles, including:
adding a tannin compound-containing natural herb medicine into water to obtain a mixture, and subjecting the mixture to heating reflux extraction to obtain a herb medicine extract; and
subjecting the herb medicine extract to fractionation to obtain the catechol nanoparticles.

In the present disclosure, unless otherwise specified, all raw materials required for preparation are commercially available products well-known to those skilled in the art.

In the present disclosure, a tannin compound-containing natural herb medicine is added into water to obtain a mixture, and the mixture is subjected to heating reflux extraction to obtain a herb medicine extract. In the present disclosure, the tannin compound-containing natural herb medicine includes one drug selected from the group consisting of Quercus infectoria Oliv., Rhus chinensis Mill., and Sanguisorba officinalis L. There is no special limitation on the source of the tannin compound-containing natural herb medicine, which may be obtained in a manner well-known in the art. In the examples of the present disclosure, the medicinal material Quercus infectoria Oliv. used was purchased from Xinjiang Ansar Pharmaceutical Co., Ltd. and stored in herbal medicine specimen library of Key Laboratory of Xinjiang Phytomedicine Resource and Utilization.

In the present disclosure, there is no special limitation on the preparation process of the tannin compound-containing natural herb medicine, and a powder may be prepared according to the well-known process in the art.

In some embodiments of the present disclosure, the water is ultrapure water. There is no special limitation on the process of adding the tannin compound-containing natural herb medicine into water, as long as the above materials may be uniformly mixed according to the well-known process in the art.

In some embodiments of the present disclosure, a ratio of a mass of the tannin compound-containing natural herb medicine to a volume of water is 1 g: 8 mL; the heating reflux extraction is conducted by atmospheric reflux extraction or vacuum reflux extraction; the atmospheric reflux extraction is conducted at 100° C.; the atmospheric reflux extraction is conducted for 2 h; the vacuum reflux extraction is conducted at 50° C.; and the vacuum reflux extraction is conducted for 2 h. In the present disclosure, the catechol compounds in the tannin compound-containing natural herb medicine may be obtained by the heating reflux extraction.

In some embodiments of the present disclosure, after the heating reflux extraction is completed, an obtained material is filtered with sterile gauze while it is hot to obtain a filter residue and a filtrate; the filter residue is discarded, and the filtrate is collected to obtain the herb medicine extract. There is no special limitation on a process of filtering and collecting, which may be conducted according to the process well known in the art.

In the present disclosure, after herb medicine extract is obtained, the herb medicine extract is subjected to fractionation to obtain the catechol nanoparticles.

In some embodiments of the present disclosure, the fractionation is conducted by a process including the following steps: subjecting the herb medicine extract to first centrifugal separation to obtain a supernatant and the catechol nanoparticles.

In some embodiments of the present disclosure, the first centrifugal separation is conducted at a centrifugal force of 6,577 g, and the first centrifugal separation is conducted for 10 min.

In some embodiments of the present disclosure, after the first centrifugal separation is completed, an obtained precipitate is washed 3 times with ultrapure water and dried to obtain the catechol nanoparticles. In some embodiments of the present disclosure, the washing is conducted by centrifugal washing; the centrifugal washing is conducted at a centrifugal force of 6,577 g; and the centrifugal washing is conducted for 3 min. In some embodiments of the present disclosure, the drying is conducted by freeze-drying. There is no special limitation on the specific process of the freeze-drying, which may be conducted according to the process well-known in the art. In some embodiments of the present disclosure, the catechol nanoparticles are stored in a refrigerator at −20° C.

In some embodiments of the present disclosure, the fractionation further includes: subjecting the supernatant to a second centrifugal separation to obtain the catechol nanoparticles. In some embodiments of the present disclosure, the second centrifugal separation is conducted at a centrifugal force of 9,500 g, and the first centrifugal separation is conducted for 10 min. By controlling the centrifugal forces of the first centrifugal separation and the second centrifugal separation to be different (differential separation), nanoparticles with different particle size distributions may be obtained.

In some embodiments of the present disclosure, after the second centrifugal separation is completed, an obtained precipitate is washed 3 times with ultrapure water and dried to obtain the catechol nanoparticles. In some embodiments of the present disclosure, the washing is conducted by centrifugal washing; the centrifugal washing is conducted at a centrifugal force of 9,500 g; and the centrifugal washing is conducted for 5 min. In some embodiments of the present disclosure, the drying is conducted by freeze-drying. There is no special limitation on the specific process of the freeze-drying, which may be conducted according to the process well-known in the art. In some embodiments of the present disclosure, the catechol nanoparticles are stored in a refrigerator at −20° C.

In the present disclosure, the supernatant obtained from the second centrifugal separation is the second supernatant used in the following solutions.

The present disclosure further provides catechol nanoparticles prepared by the method described in the above technical solutions, where the catechol nanoparticles are formed by self-assembly of a catechol and a protein, and have an average particle size of 413.89 nm±202.95 nm or 230.34 nm±59.48 nm. In the present disclosure, the catechol compounds in the herb medicine self-assemble with proteins existing in the herb medicine to form a catechol protein nanocomposite.

The present disclosure further provides a method for preparing a Quercus infectoria Oliv. catechol protein nanoparticles, including:
preparing a herb medicine extract by the method described in the above technical solutions; and
subjecting the herb medicine extract to first centrifugal separation to obtain a first supernatant, subjecting the first supernatant to second centrifugal separation to obtain a second supernatant, mixing the second supernatant, water, and a protein to obtain a mixture, subjecting the mixture to self-assembly under heating and conducting separation to obtain the catechol protein nanoparticles.

There is no special limitation on the processes of the preparation of the herb medicine extract, the first centrifugal separation, and the second centrifugal separation, which may be conducted according to the process described in the above technical solutions.

In some embodiments of the present disclosure, the herb medicine extract in the second supernatant has a concentration of 10 mg/mL.

In some embodiments of the present disclosure, the water is ultrapure water; a ratio of a volume of water to a mass of the second supernatant is 60 mL:600 mg.

In the present disclosure, the protein includes one protein selected from the group consisting of bovine serum albumin (BSA), lysozyme (LYZ), cytochrome C (CYC), β-lactoglobulin (bLG), pepsin, β-galactosidase (β-gal), hemoglobin (Hgb), fibrinogen (FGN), immunoglobulin G (IgG), horseradish peroxidase (HRP), and glucose oxidase (GOX); and a mass ratio of the protein to the second supernatant is in a range of (10-300):600.

There is no special limitation on the process of mixing the second supernatant, water, with protein, which may be conducted according to processes well-known in the art.

In some embodiments of the present disclosure, the heating is conducted at 100° C.; the self-assembly is conducted for 2 h; the heating is conducted under stirring; and the heating is conducted at a speed of 200 rpm.

In some embodiments of the present disclosure, the separation is conducted by centrifugal separation; the centrifugal separation is conducted at a centrifugal force of 5,000 g; and the centrifugal separation is conducted for 10 min.

In some embodiments of the present disclosure, after the separation is completed, an obtained precipitate is washed 3 times and dried to obtain the Quercus infectoria Oliv. catechol protein nanoparticles; the washing is conducted by centrifugal washing; the centrifugal washing is conducted at a centrifugal force of 5,000 g; and the centrifugal separation is conducted for 5 min. In some embodiments of the present disclosure, the drying is conducted by freeze-drying. There is no special limitation on the specific process of the freeze-drying, which may be conducted according to the process well-known in the art. In some embodiments of the present disclosure, the catechol protein nanoparticles are stored in a refrigerator at −20° C.

The present disclosure further provides catechol protein nanoparticles prepared by the method described in the above technical solutions.

The present disclosure further provides use of the catechol nanoparticles described in the above technical solutions or the catechol protein nanoparticles described in the above technical solutions as a drug carrier. There is no special limitation on the method of use, and methods well-known in the art may be used.

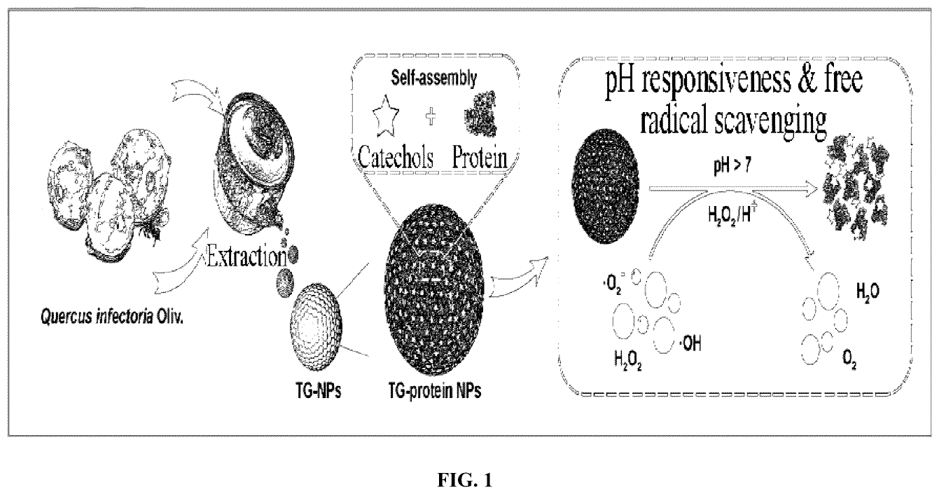

FIG. 1 is a schematic diagram showing the process, mechanism for preparation of the catechol nanoparticles according to an embodiment of the present disclosure and the performance of the catechol nanoparticles. In the present disclosure, the natural catechol nanoparticles can be obtained after separating the herb medicine extract (taking Quercus infectoria Oliv. as an example). A formation mechanism of the nanoparticles is the self-assembly of catechol compounds and trace proteins in the herb medicine extract. The catechol nanoparticles prepared have pH responsiveness and the ability to scavenge free radicals.

The technical solutions of the present disclosure will be clearly and completely described below with reference to the examples of the present disclosure. Apparently, the described examples are merely a part rather than all of the examples of the present disclosure. All other embodiments obtained by those skilled in the art based on the embodiments of the present disclosure without creative efforts shall fall within the protection scope of the present disclosure.

In the following examples, the source of reagents used are as follows:

Acetonitrile, chromatographic grade, was purchased from Thermo Fisher Scientific Corporation, USA; formic acid, excellent grade, was purchased from Tianjin Guangfu Fine Chemical Research Institute; 1,1-diphenyl-2-trinitrophenylhydrazine (DPPH) was purchased from Sigma-Aldrich; 2,2′-azino-bis-3-ethylbenzothiazoline-6-sulfonic acid (ABTS) was purchased from Shanghai Lanji Biological Co., Ltd. K₂S₂O₈ was purchased from Shanghai McLean Biochemical Co., Ltd.; vitamin C (VC), bovine serum albumin (BSA), and lysozyme (LYZ) were purchased from Beijing Solarbio Science & Technology Co., Ltd. Glucose oxidase (GOX), hemoglobin (Hgb), immunoglobulin G (IgG), fibrinogen (FGN), horseradish peroxidase (HRP), cytochrome C (CYC), and β-galactosidase (β-gal) were purchased from Shanghai Yuanye Bio-Technology Co., Ltd., China.

### Example 1

A crude powder of Quercus infectoria Oliv. (40.00 g) was added to 8 times an amount of ultrapure water (320.00 mL), obtaining a mixture. The mixture was subjected to reflex extraction at 100° C. for 2 h, filtered with sterile gauze while it was hot, obtaining a filter residue and a filtrate. The filter residue was discarded. The filtrate was collected, obtaining a Quercus infectoria Oliv. extract (TGE).

The Quercus infectoria Oliv. extract TGE was centrifuged (at a centrifugal force of 6,577 g for 10 min), obtaining a first supernatant and a first precipitate. The first supernatant was collected and marked as TG-LS. The first precipitate was washed 3 times with ultrapure water (at a centrifugal force of 6,577 g for 3 min), and marked as TG-LP NPs. Then the TG-LS was centrifuged (at a centrifugal force of 9,500 g for 10 min), obtaining a second supernatant and a second precipitate. The second supernatant was collected, and labeled as TG-HS. The second precipitate was washed 3 times with ultrapure water (at a centrifugal force of 9,500 g for 5 min), and labeled as TG-HP NPs. The supernatant (TG-HS) and the precipitates (TG-LP NPs and TG-HP NPs) were separately freeze-dried and stored in a −20° C. refrigerator.

Characterization

1) Scanning Electron Microscopy (SEM)

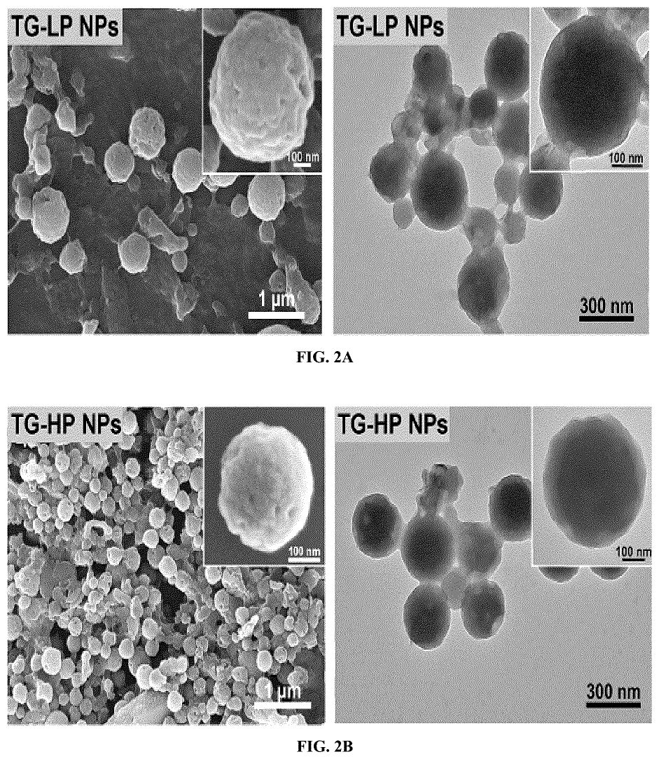

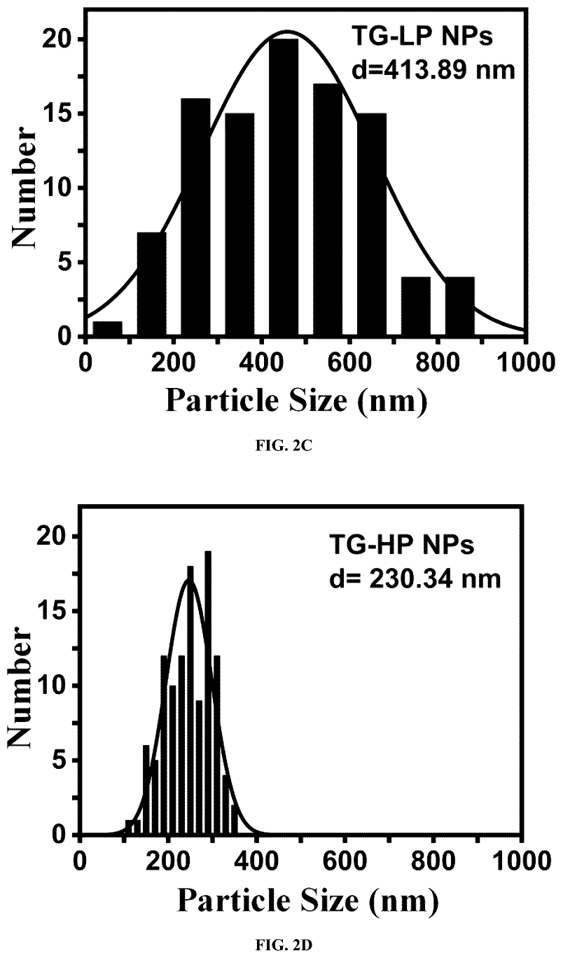

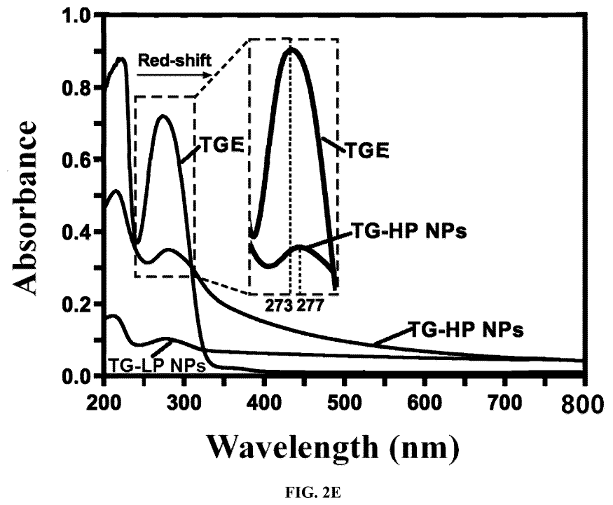

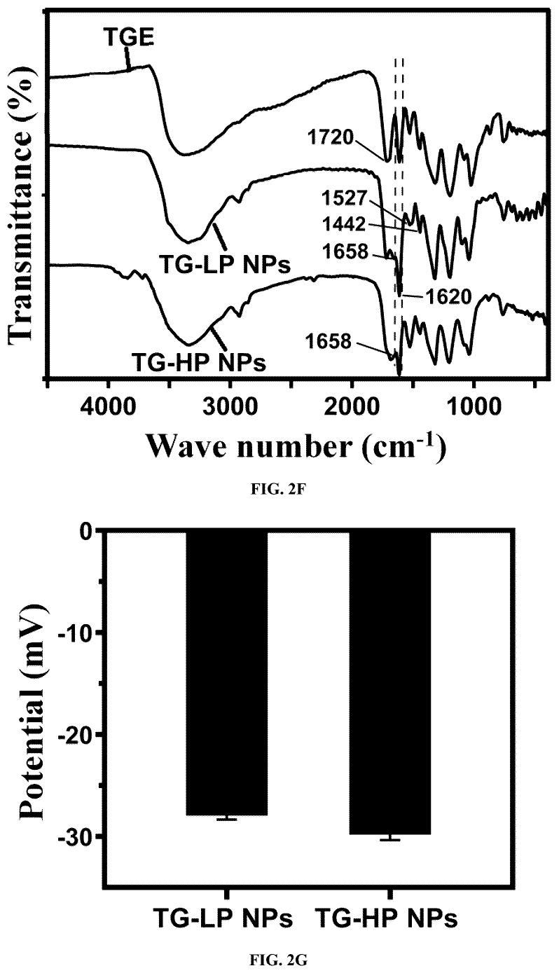

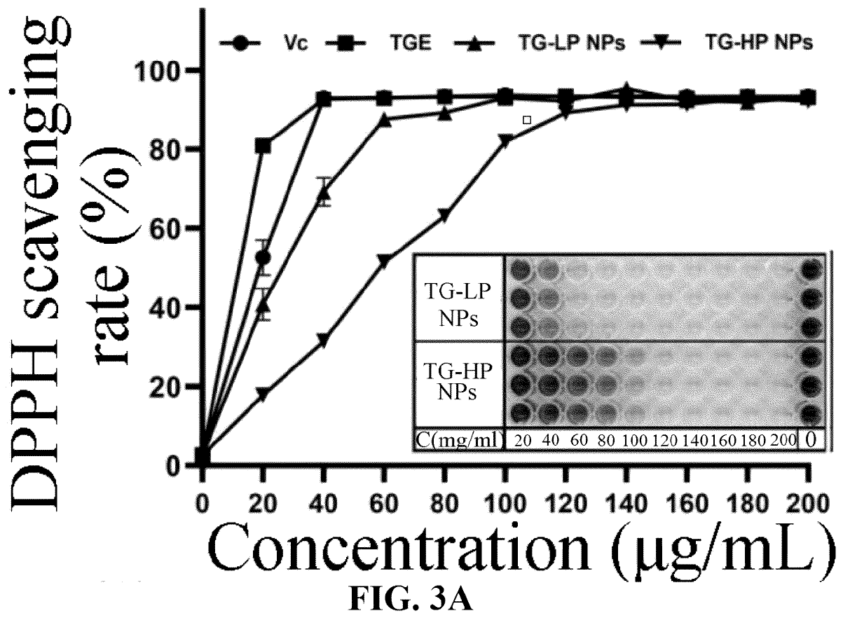

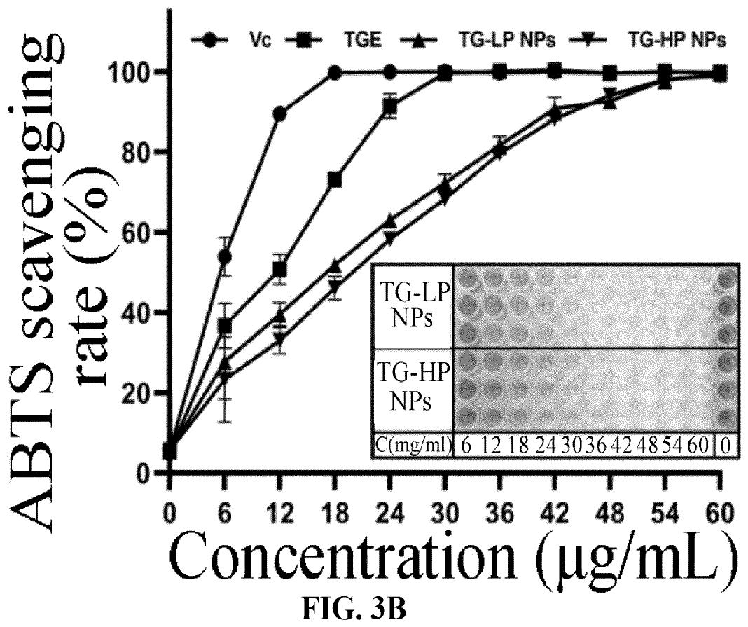

The Quercus infectoria Oliv. catechol nanoparticles TG-LP NPs and TG-HP NPs prepared in Example 1 were uniformly dispersed in ultrapure water, respectively, dropwise added onto a silicon wafer, obtaining a silicon wafer loaded with Quercus infectoria Oliv. catechol nanoparticles. The silicon wafer loaded with Quercus infectoria Oliv. catechol nanoparticles was dried, and sprayed with gold. Then the morphology and particle size of the Quercus infectoria Oliv. catechol nanoparticles were observed by SEM. 100 nanoparticles were randomly selected by Image J software for particle size measurement. The results are shown in FIG. 2.

2) Transmission Electron Microscopy (TEM)

The Quercus infectoria Oliv. catechol nanoparticles TG-LP NPs and TG-HP NPs prepared in Example 1 were uniformly dispersed in ultrapure water, respectively, dropwise added onto a copper grid, obtaining a copper grid loaded with Quercus infectoria Oliv. catechol nanoparticles. The copper grid loaded with Quercus infectoria Oliv. catechol nanoparticles was dried. Then the morphology of the Quercus infectoria Oliv. catechol nanoparticles was observed by TEM. The results are shown in FIG. 2.

3) UV-Vis Absorption Spectrum

The Quercus infectoria Oliv. extract TGE, Quercus infectoria Oliv. catechol nanoparticles TG-LP NPs and TG-HP NPs were evenly dispersed in ultrapure water, and their spectra were scanned at a wavelength of 200 nm to 800 nm. The results are shown in FIG. 2.

4) Fourier Transform Infrared Spectroscopy

Equal amounts of freeze-dried TGE, TG-LP NPs, and TG-HP NPs powders were separately mixed with a dried potassium bromide (KBr) powder in an agate mortar, obtaining a mixed powder. The mixed powder was fully ground until the particle size was less than 2 μm. An appropriate amount of sample was put into a tableting mold, and pressed into a transparent sheet on a tablet machine. The functional group characteristics of the sample were measured at a range of 4,500 cm⁻¹ to 500 cm⁻¹ by Fourier transform infrared spectrometer. The results are shown in FIG. 2.

5) Potential analysis was conducted on the Quercus infectoria Oliv. catechol nanoparticles prepared in Example 1. The results are shown in FIG. 2.

FIG. 2A to FIG. 2G show characterization results of the Quercus infectoria Oliv. catechol nanoparticles TG-LP NPs and TG-HP NPs prepared in Example 1; where FIG. 2A represents SEM (left) and TEM (right) images of TG-LP nanoparticles; FIG. 2B represents SEM (left) and TEM (right) images of TG-HP NPs, inset: the representative appearance of nanoparticles; FIG. 2C represents a particle size distribution of TG-LP NPs; FIG. 2D represents a particle size distribution of TG-HP NPs; FIG. 2E represents a UV-visible absorption spectrum of TGE, TG-LP NPs, and TG-HP NPs at a same concentration; FIG. 2F represents a Fourier transform infrared spectrum of TGE, TG-LP NPs, and TG-HP NPs; and FIG. 2G represents a Zeta potential diagram of Quercus infectoria Oliv. catechol nanoparticles TG-LP NPs and TG-HP NPs.

FIG. 2A and FIG. 2B represent the appearance images of Quercus infectoria Oliv. catechol nanoparticles TG-LP NPs and TG-HP NPs, and the inset is a higher magnification image. As shown in FIG. 2A and FIG. 2B, the Quercus infectoria Oliv. catechol nanoparticles TG-LP NPs and TG-HP NPs can be fractionated from the Quercus infectoria Oliv. extract by differential centrifugation, and their surfaces are rough and even have some holes.

FIG. 2C and FIG. 2D represent the particle size distribution graphs of Quercus infectoria Oliv. catechol nanoparticles, showing that the Quercus infectoria Oliv. catechol nanoparticles TG-LP NPs have an average particle size of 413.89 nm±202.95 nm, and the TG-HP NPs have an average particle size of 230.34 nm±59.48 nm. This shows that with the increase of centrifugal force, the average particle size of TG-NPs decreases and the particle size becomes more uniform.

FIG. 2E represents a UV-vis spectrum of TGE, TG-LP NPs, and TG-HP NPs. As shown in FIG. 2E, TGE, TG-LP NPs, and TG-HP NPs show similar characteristic absorption peaks of Quercus infectoria Oliv. catechol (at around 270 nm).

FIG. 2F shows the FT-IR spectra of TGE, TG-LP NPs, and TG-HP NPs, illustrating stretching vibrations of the aromatic ring (C—C/C═C) at 1,527, 1,442, and 1,620 cm⁻¹. The absorption at 1,720 cm⁻¹ belongs to the stretching vibration of aromatic esters (C═O), indicating that the nanoparticles are rich in Quercus infectoria Oliv. Catechol compounds or their derivatives. The absorption peaks at 1,658 cm⁻¹ of TG-LP NPs and TG-HP NPs indicate that there may be amide groups in the nanoparticles. These results clearly indicate that the chemical composition of the nanoparticles may have catechol-like structures and amide groups.

FIG. 2G is a potential diagram of the Quercus infectoria Oliv. catechol nanoparticles prepared in Example 1. As shown in FIG. 2G, the potential values of Quercus infectoria Oliv. catechol nanoparticles TG-LP NPs and TG-HP NPs are −27.97 mV and −29.83 mV, respectively, indicating that these two nanoparticles have desirable stability.

Performance Testing

### 1. Antioxidant Activity of TGE, Quercus infectoria Oliv. Catechol Nanoparticles (TG-LP NPs and TG-HP NPs) Prepared in Example 1

1) Determination of DPPH Scavenging Rate

19.7 mg of DPPH was precisely weighed, added into 25 mL of methanol, and configured as a DPPH working solution with a concentration of 2 mM. 2 mL of the working solution was sucked up, mixed with 18 mL of methanol, and diluted to 20 mL, obtaining a DPPH working solution with a concentration of 0.2 mM. Then the DPPH working solution was stored in a refrigerator at 4° C. in the dark. 150 μL of a sample to be tested and 75 μL of the DPPH working solution were sucked up, mixed to even, and reacted at room temperature in the dark for 30 min. An absorbance at 517 nm was measured using a microplate reader. Each sample was tested 3 times in parallel. A calculation formula of DPPH. scavenging rate was:

$$DPPH \cdot scavenging rate = ( 1 - \frac{{Abs}_{1} - {Abs}_{2}}{{Abs}_{0}} ) \times 100 \% \qquad ( 2 - 1 )$$

In Formula 2-1, Abs₀ represents the absorbance of a blank control group (deionized water replacing the sample to be tested), Abs₁ represents the absorbance value of the sample to be tested, and Abs₂ represents the absorbance of a sample control (methanol solution replacing the DPPH working solution).

2) Determination of ABTS⁺ Scavenging Rate

33 mg of K₂S₂O₈ was precisely weighed, dissolved in a small amount of deionized water, diluted to 50.00 mL, and configured as a K₂S₂O₈ solution with a concentration of 2.45 mM. 38.00 mg of ABTS was precisely weighed, and dissolved in a small amount of deionized water, diluted to 10 mL, and configured as an ABTS solution with a concentration of 7.0 mM. 10 mL each of the K₂S₂O₈ solution and the ABTS solution were mixed to even, incubated at room temperature in the dark for 12 h, and diluted with methanol such that its absorbance at 734 nm was 0.70±0.02, obtaining an ABTS free radical working solution. 50 μL of a sample to be tested and 150 μL of the ABTS free radical working solution were sucked up and mixed to even, and reacted at room temperature in the dark for 6 min. An absorbance at 734 nm was measured using a microplate reader. Each sample was tested 3 times in parallel, and a calculation formula of ABTS⁺ scavenging rate was as follows:

$${ABTS}^{+} scavenging rate = ( 1 - \frac{{Abs}_{1} - {Abs}_{2}}{{Abs}_{0}} ) \times 100 \% \qquad ( 2 - 2 )$$

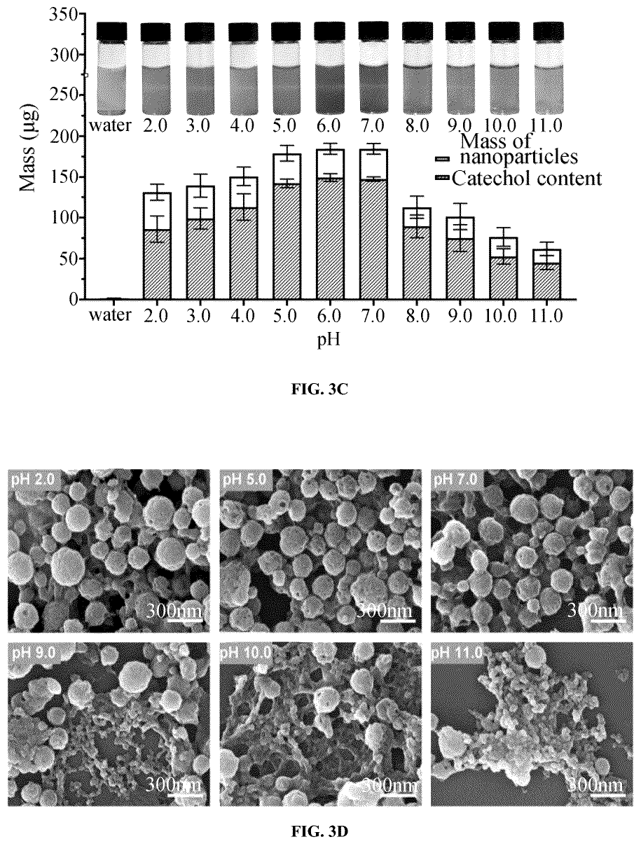

According to the above determination steps, VC (Vitamin C) was used as a control sample for comparison. The obtained antioxidant activity results are shown in FIG. 3A to FIG. 3B. The antioxidant activity of Quercus infectoria Oliv. catechol nanoparticles (TG-LP NPs and TG-HP NPs) prepared in Example 1 was evaluated by DPPH and ABTS free radical scavenging experiments. The inset visually showed the results. The free radical scavenging efficiency of TG-LP NPs and TG-HP NPs is concentration-dependent, and the color of DPPH and ABTS solutions gradually fades with the increase in concentration. Compared with the VC and Quercus infectoria Oliv. extract TGE, the Quercus infectoria Oliv. catechol nanoparticles TG-LP NPs and TG-HP NPs have reduced antioxidant activity, but still retain their original antioxidant activity.

### 2. pH Responsiveness of the Quercus infectoria Oliv. Catechol Nanoparticles Prepared in Example 1

1) 2.00 mg of TG-HP NPs prepared in Example 1 were placed in 4.00 mL of different buffer solutions with pH values of 2.0, 3.0, 4.0, 5.0, 6.0, 7.0, 8.0, 9.0, 10.0, and 11.0 (potassium chloride-hydrochloric acid buffer was used when pH was 2.0; citric acid-sodium citrate buffer was used when pH was 3.0, 4.0, or 5.0; phosphate buffer was used when pH was 6.0, 7.0, or 8.0; tris-HCl buffer was used when pH was 9.0; and sodium bicarbonate-sodium hydroxide buffer was used when pH was 10.0 or 11.0). The resulting solution was vigorously mixed with a vortex mixer for 1 min, shaken in a constant-temperature oscillator for 30 min (at a speed of 200 rpm, at 37° C.), and then taken out to observe the color change thereof and whether there was a Tyndall effect. The mixed solution was centrifuged (at a centrifugal force of 9,500 g for 15 min), obtaining a precipitate. The precipitate was collected and dispersed in deionized water, and dropped onto a silicon chip, obtaining a silicon chip loaded with the precipitate. The silicon chip loaded with the precipitate was dried and sprayed with gold. The morphology changes of Quercus infectoria Oliv. catechol nanoparticles were observed by SEM when the pH values were 2.0, 5.0, 7.0, 9.0, 10.0, and 11.0, respectively.

FIG. 3C represents a graph showing the change in intensity of the Tyndall effect after co-incubation and shaking of Quercus infectoria Oliv. catechol nanoparticles with water and buffer solutions with different pH values for 30 min. It can be seen that when the pH values are 8.0, 9.0, 10.0, and 11.0, the red light intensity of the Tyndall effect is obviously weakened.

FIG. 3D represents an SEM image of Quercus infectoria Oliv. catechol nanoparticles co-incubated and shaken with buffer solutions with different pH values for 30 min. It can be clearly seen that a large number of TG-HP NPs have broken down into small particles after the nanoparticles are shaken with pH 9.0, pH 10.0, and pH 11.0 buffers; when the pH value is 2.0, 5.0 and 7.0, the appearance of the nanoparticles does not change significantly.

2) Determination of Content Changes of Catechol by Folin Reagent Method

A. Establishment of a Standard Curve for Gallic Acid (GA)

10.00 mg of a GA reference substance was put into a 50 mL brown measuring bottle, mixed with water to dissolve, and diluted to a mark, obtaining a mixed solution. The mixed solution was shaken to even, obtaining a reference solution (every 1 mL contained 0.2 mg of GA).

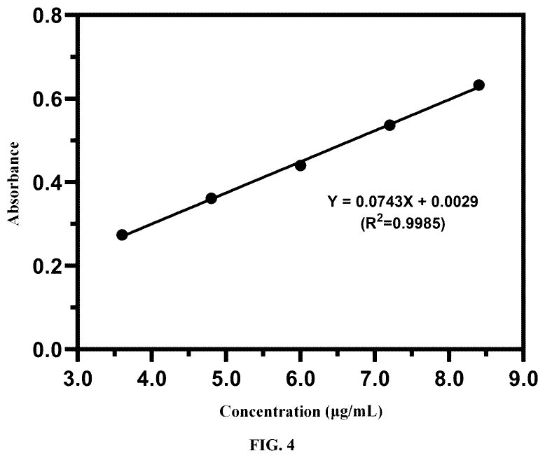

100 μL, 150 μL, 200 μL, 250 μL, 300 μL and 350 μL of the reference solution were precisely weighed, and put into a 10 mL brown volumetric flask separately. 0.5 mL of Folin reagent was added to the brown volumetric flask, and shaken to even. 2 mL of 15% sodium carbonate solution was added to the resulting solution within 2 min, diluted with water to a mark. The brown volumetric flask was tightened with a stopper, and shaken to even. A corresponding reagent was used as a blank. The samples were transferred to a brown test tube with a stopper, put in a water bath at 70° C. for 15 min, rapidly cooled, and stood for 10 min. An absorbance was measured at a wavelength of 760 nm according to UV-Vis spectrophotometry (general rule 0401). With the absorbance as an ordinate and the concentration as an abscissa, a standard curve was plotted, as shown in FIG. 4.

B. Determination of the catechol content change of TG-HP NPs in different pH buffer solutions: The Quercus infectoria Oliv. catechol nanoparticles TG-HP NPs prepared in Example 1 were co-incubated with buffer solutions of different pH values, obtaining a mixed solution. The mixed solution was centrifuged (at a centrifugal force of 9,500 g for 15 min), obtaining a precipitate. The precipitate was resuspended by 1 mL of ultrapure water. 250 μL of the resuspended solution was added to a 10 mL brown measuring bottle. An absorbance was measured according to the method in the preparation of the standard curve (method in A above), starting from “adding 0.5 mL of Folin reagent”. The amount (μg) of Quercus infectoria Oliv. catechol in TG-HP NPs in different pH buffer solutions was measured according to the standard curve. The results are shown in FIG. 3C.

In FIG. 3C represents the change of the mass and polyphenol content of the nanoparticles with the pH value after the co-incubation and shaking of the Quercus infectoria Oliv. catechol nanoparticles with water and buffer solutions of different pH values for 30 min. It is seen that when the pH value is greater than 7.0, the mass and polyphenol content of nanoparticles are significantly reduced.

The results in FIG. 3C to FIG. 3D both illustrate that the TG-HP NPs prepared by the present disclosure are more prone to degradation under alkaline conditions (pH>7.0).

Composition Analysis of Quercus infectoria Oliv. Catechol Nanoparticles

1) Component analysis: TGE, Quercus infectoria Oliv. catechol nanoparticles TG-LP NPs and TG-HP NPs prepared in Example 1 were analyzed by high-performance liquid chromatography-mass spectrometry (LC-MS). TGE, TG-LP NPs, and TG-HP NPs were diluted with ultrapure water to a concentration of 10 mg/mL, and passed through a 0.22 pin filter membrane. 20 μL of sample was injected into a high-performance liquid chromatography system. A mobile phase consisted of a solvent A (distilled water/0.2% formic acid, 499:1, v/v) and a solvent B (acetonitrile). Compounds were separated using a gradient program: 0 min to 4 min (93% to 93% A), 4 min to 8 min (93% to 90% A), 8 min to 40 min (90% to 80% A), 40 min to 50 min (80% to 70% A), and 50 min to 65 min (70% to 0% A), and the flow rate was 1 mL/min.

The detection wavelengths of the UV detector were 254 nm and 269 nm; ESI-MS conditions: negative ion mode; scanning range: 100-1250 (m/z); ion source temperature: 120° C.; desolvation temperature: 250° C.; capillary voltage: 2.8 kV; cone voltage: 50 V.

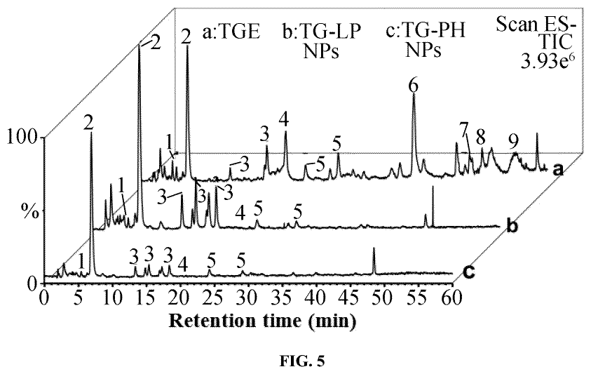

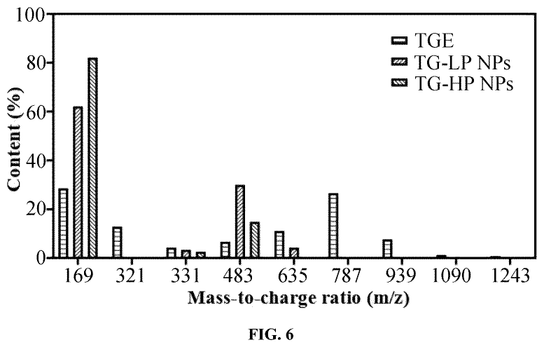

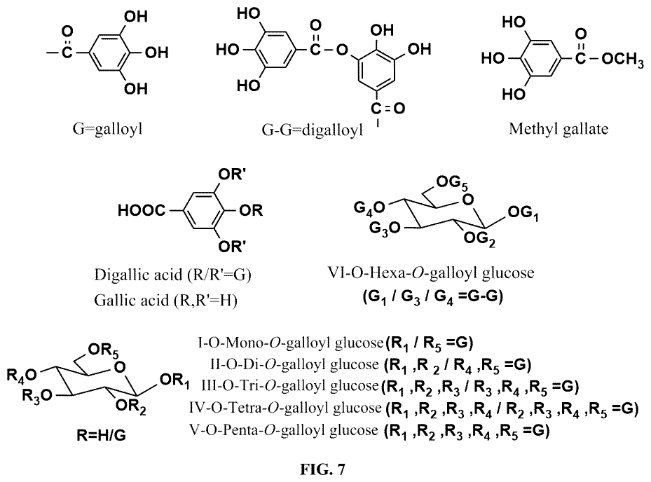

According to the mass spectrum ion fragments and compound information of substances with different molecular weights, the basic material composition information of nanosphere TG-NPs was inferred. FIG. 5 shows the results of the chemical composition of TGE, TG-LP NPs, and TG-HP NPs measured by LC-MS. After analysis and identification, it is confirmed that gallopolyphenol nanospheres containe 2 main components and 9 compounds, namely gallic acid and phenolic acid (gallic acid and m-digallic acid) and gallic acid tannins (including mono-O-galloyl glucose, di-O-galloyl glucose, tri-O-galloyl glucose, tetra-O-galloyl glucose, penta-O-galloyl glucose, hexa-O-galloyl glucose, and hepta-O-galloyl glucose). The specific mass spectrometry information and identification results are shown in Table 1.

TABLE 1
Chemical composition, retention time, mass-to-charge
ratio, and peak area of TGE, TG-LP NPs, and TG-HP NPs

| | | | Mass-to- | Retention time (min) | | | Peak area (%) | | |
|---|---|---|---|---|---|---|---|---|---|
| Peak | Molecular | | charge | | TG-LP | TG-HP | | TG-LP | TG-HP |
| SN | formula | Component | ratio (m/z) | TGE | NPs | NPs | TGE | NPs | NPs |
| 1 | C₁₃H₁₆O₁₀ | Mono-O- | 331 | 4.87, | 3.59, | 3.52, | 4.3 | 3.39 | 2.57 |
| | | galloyl | | 6.40, | 4.34, | 4.74, | | | |
| | | glucose | | 9.63 | 5.32, | 5.26, | | | |
| | | | | | 6.33 | 6.15 | | | |
| 2 | C₇H₆O₅ | Gallic | 169 | 7.06 | 6.80, | 6.84, | 28.58 | 62.13 | 82.10 |
| | | acid | | | 6.92, | 7.02 | | | |
| | | | | | 7.14 | | | | |
| 3 | C₂₀H₁₉O₁₄ | Di-O- | 483 | 10.26, | 10.14, | 13.40, | 6.72 | 29.98 | 14.87 |
| | | galloyl | | 13.34, | 13.19, | 15.35, | | | |
| | | glucose | | 18.45, | 15.27, | 18.25, | | | |
| | | | | 18.72 | 18.31 | | | | |
| 4 | C₁₄H₁₀O₉ | M-digallic | 321 | 18.81, | 21.39 | 25.98 | 12.85 | 0.03 | 0.04 |
| | | acid | | 21.57 | | | | | |
| 5 | C₂₇H₂₃O₁₈ | Tri-O- | 635 | 24.40, | 24.26, | 29.38 | 11.14 | 4.30 | 0.06 |
| | | galloyl | | 29.21, | 28.85, | | | | |
| | | glucose | | 31.41, | 30.01, | | | | |
| | | | | 32.96, | 34.76, | | | | |
| 6 | C₃₄H₂₇O₂₂ | Tetra-O- | 787 | 32.14, | 41.68 | 48.85 | 26.62 | 0.03 | 0.07 |
| | | galloyl | | 38.34, | | | | | |
| | | glucose | | 40.31, | | | | | |
| | | | | 41.39 | | | | | |
| 7 | C₄₁H₃₁O₂₆ | Penta-O- | 939 | 46.65, | 26.34, | 28.51 | 7.66 | 0.04 | 0.08 |
| | | galloyl | | 48.54 | 38.49 | | | | |
| | | glucose | | | | | | | |
| 8 | C₄₈H₃₅O₃₀ | Hexa-O- | 1091 | 51.38, | 10.61, | 9.58, | 1.29 | 0.05 | 0.11 |
| | | galloyl | | 55.35 | 22.59, | 31.35, | | | |
| | | glucose | | | 26.37, | 42.38 | | | |
| | | | | | 35.75 | | | | |
| 9 | C₅₅H₃₉O₃₄ | Hepta-O- | 1243 | 54.67, | 32.65, | 22.24 | 0.83 | 0.05 | 0.12 |
| | | galloyl | | 55.37 | 38.49 | | | | |
| | | glucose | | | | | | | |

FIG. 6 shows a heat map comparing the content changes of main components in different nanoparticles by calculating the peak area. It is clearly seen that the peak areas of compounds with mass-to-charge ratios greater than 635 are significantly reduced after TGE is prepared into nanoparticles. This indicates that Quercus infectoria Oliv. catechol nanoparticles TG-LP NPs and TG-HP NPs are mainly composed of Quercus infectoria Oliv. catechol oligomers with a smaller molecular weight (mass-to-charge ratio <635). FIG. 7 shows chemical structural formulas of 9 kinds of catechol series in Quercus infectoria Oliv.

2) Elemental Analysis of Nanoparticles

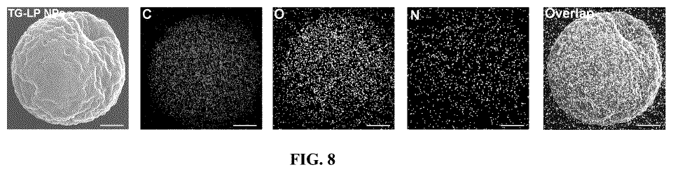

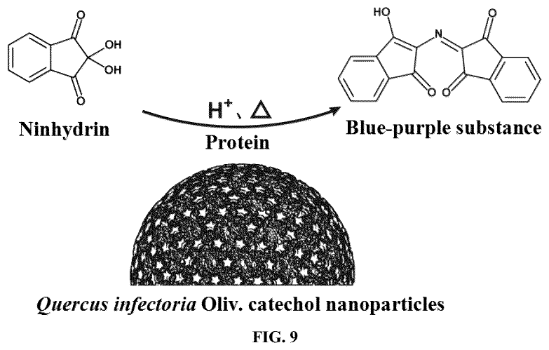

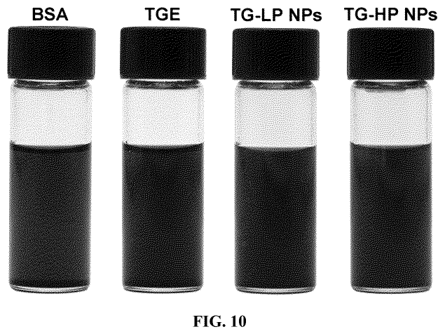

An appropriate amount of TG-LP NPs was uniformly dispersed in ultrapure water, dropped onto a silicon wafer, obtaining a silicon wafer loaded with TG-LP NPs. The silicon wafer loaded with TG-LP NPs was dried and sprayed with gold. The morphology and elemental analysis of the TG-LP NPs was observed by SEM. The results are shown in FIG. 8. FIG. 8 shows an energy dispersive X-ray (EDX) analysis diagram of the TG-LP NPs, and the scale bar is 200 nm. The results shows that C, O, and N elements exist in TG-LP NPs. The presence of N element suggests that the formation of Quercus infectoria Oliv. catechol nanoparticles is probably related to the insect galls containing a small amount of protein in the Quercus infectoria Oliv Quercus infectoria Oliv. is a dried insect gall formed by the parasitism of insect gall wasps on Quercus infectoria G. Olivier. Quercus infectoria Oliv. is the dried insect gall that form on trees. In addition, they all contain rich catechol structures. Therefore, it is inferred that the formation of regular nanoparticles in the Quercus infectoria Oliv. extract is actually formed by the self-assembly of catechol compounds and proteins with a molecular weight less than or equal to 635.

3) Ninhydrin Color Reaction

10.00 mg of BSA, dried TGE, TG-LP NPs, and TG-HP NPs were weighed separately, mixed with 5.00 mL of ninhydrin chromogenic solution (1.50 g of ninhydrin powder+100.00 mL of n-butanol+3.00 mL of glacial acetic acid), stirred to even, and heated in a boiling water bath for 10 min. The color change was observed, and pictures were taken in time.

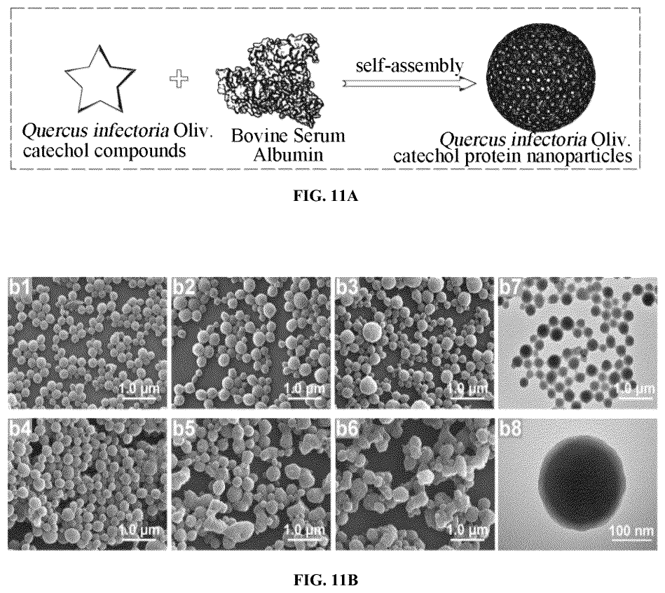

FIG. 9 is a schematic diagram of the ninhydrin reaction. Ninhydrin reaction means that α-amino acid and all proteins can react with ninhydrin to generate blue-purple substances under heating in a weak acid environment.

FIG. 10 is a solution picture after adding a ninhydrin chromogenic solution in the positive control group (BSA) and sample groups (Quercus infectoria Oliv. extract TGE, Quercus infectoria Oliv. catechol nanoparticles TG-LP NPs and TG-HP NPs). It can be seen that the control group is a blue-purple solution, and the sample group is a purple-black solution. This is because the TGE, TG-LP NPs, and TG-HP NPs themselves are yellow-brown, such that a final overlay color is black-purple. This result indicates that the proteins exist in TGE, TG-LP NPs, and TG-HP NPs.

The results of chemical composition identification, elemental analysis, and ninhydrin color reaction indicate that the nanoparticles with regular shapes in Quercus infectoria Oliv. are formed by self-assembly of catechol compounds with a smaller molecular weight (mass-to-charge ratio <635) and proteins.

### Example 2

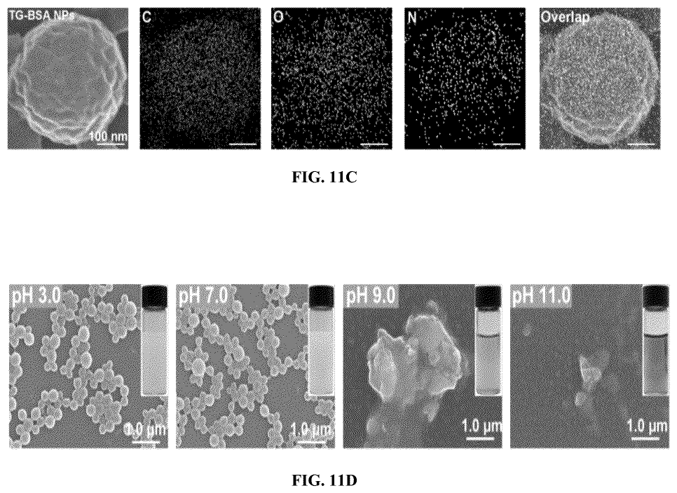

600.00 mg of the TG-HS solution (with a concentration of 10 mg/mL) prepared in Example 1 was precisely weighed and added to 60.00 mL of ultrapure water, obtaining a mixed solution. The mixed solution was stirred to even. Then different amounts of BSA (10.00 mg, 30.00 mg, 50.00 mg, 100.00 mg, 200.00 mg, 300.00 mg, 400.00 mg, 500.00 mg, 600.00 mg) were added to the mixed solution, obtaining a mixture. The mixture was stirred (at a speed of 200 rpm for 2 h) with heating reflux at 100° C., and subjected to centrifugation (at a centrifugal force of 5,000 g for 10 min), collecting a precipitate. The precipitate was washed 3 times with water (at a centrifugal force of 5,000 g for 5 min), obtaining a supernatant and a nanoparticles. The supernatant was discarded. The nanoparticles were marked as TG-BSA-1 NPs, TG-BSA-2 NPs, TG-BSA-3 NPs, TG-BSA-4 NPs, TG-BSA-5 NPs, TG-BSA-6 NPs, TG-BSA-7 NPs, TG-BSA-8 NPs, and TG-BSA-9 NPs, freeze-dried, and stored in a −20° C. refrigerator.
1) FIG. 11A represents a schematic diagram of the reaction process between Quercus infectoria Oliv. catechol compounds and BSA; different feeding amounts of BSA were used to react with TG-HS solution. The specific parameters and the mass of the obtained nanoparticles are shown in Table 2:

TABLE 2
Synthesis parameters of Quercus infectoria Oliv. catechol protein nanoparticles TG-BSA NPs

| | TG- | TG- | TG- | TG- | TG- | TG- | TG- | TG- | TG- |
|---|---|---|---|---|---|---|---|---|---|
| | BSA-1 | BSA-2 | BSA-3 | BSA-4 | BSA-5 | BSA-6 | BSA-7 | BSA-8 | BSA-9 |
| Name | NPs | NPs | NPs | NPs | NPs | NPs | NPs | NPs | NPs |
| TG-HS (mg) | 600 | 600 | 600 | 600 | 600 | 600 | 600 | 600 | 600 |
| BSA (mg) | 10 | 30 | 60 | 100 | 200 | 300 | 400 | 500 | 600 |
| TG-BSA | 16.4 | 82.0 | 115.6 | 256.4 | 524.0 | 642.4 | 679.8 | 684.1 | 683.9 |
| NPs (mg) | | | | | | | | | |

Table 2 shows the mass results of the nanoparticles obtained after feeding different amounts of BSA. As shown in Table 2, with the continuous addition of protein, more and more nanoparticles are obtained. However, when the feeding amount of TG-HS is greater than 600 mg and the feeding amount of BSA is greater than 400 mg, the amount of obtained nanoparticles does not increase significantly. This indicates that when the feeding amount of BSA is greater than 400 mg, the catechol compounds of TG-HS have been fully consumed. Therefore, even if more BSA is added, the yield of nanoparticles does increase anymore.
2) Determination of the pH responsiveness of the Quercus infectoria Oliv. protein nanoparticles prepared in Example 2:

4.00 mg of TG-BSA NPs were placed in 4 mL of different buffer solutions with pH values of 3.0, 7.0, 9.0, and 11.0 (citric acid-sodium citrate buffer was used when pH was 3.0; phosphate buffer was used when pH was 7.0; tris-HCl buffer was used when pH was 9.0; sodium bicarbonate-sodium hydroxide buffer was used when pH was 11.0). The resulting solution was vigorously mixed with a vortex mixer for 1 min, shaken in a constant-temperature oscillator for 30 min (at a speed of 200 rpm, at 37° C.), and then taken out to observe the color change thereof. The mixed solution was centrifuged (at a centrifugal force of 9,500 g for 15 min), obtaining a precipitate. The precipitate was collected and dispersed in deionized water, and dropped onto a silicon wafer, obtaining a silicon wafer loaded with the precipitate. The silicon wafer loaded with the precipitate was dried and sprayed with gold. The morphology changes of Quercus infectoria Oliv. catechol nanoparticles were observed by SEM. The results are shown in FIG. 11D.

FIG. 11D represents SEM images of the Quercus infectoria Oliv. catechol protein nanoparticles TG-BSA-2 NPs (30 mg of BSA) prepared in Example 2 incubated with different pH buffer solutions for 30 min; inset represents photos of TG-BSA NPs added to different pH buffer solutions (3.0, 7.0, 9.0, and 11.0). This indicates that TG-BSA NPs also show obvious pH responsiveness. When TG-BSA NPs are mixed with buffers of different pH values (3.0, 7.0, 9.0, and 11.0), the color of the solutions changes accordingly. In alkaline buffer solutions with pH values of 9.0 and 11.0, the color of the solutions rapidly changes from cloudy milky white to a clear yellowish brown. The SEM images show that when the pH value is 3.0 or 7.0, the nanoparticles have little change in shape and are still spherical nanoparticles with regular shapes. When the pH values are 9.0 and 11.0, the nanoparticles deform or even disappear.
3) FIG. 11B represents an SEM image of TG-HS solution and TG-BSA NPs formed by adding different BSA contents (b1 to b6 corresponding to: 10.00 mg, 30.00 mg, 50.00 mg, 100.00 mg, 200.00 mg, 300.00 mg); b7 and b8 represent TEM images of TG-BSA NPs with BSA feeding amount of 30.00 mg under different magnification conditions. This illustrates that the method can also obtain a large number of spherical nanoparticles with regular shapes, and is extremely similar to the shape of nanoparticles existing in the Quercus infectoria Oliv. extract.
4) FIG. 11C represents an EDX analysis diagram of Quercus infectoria Oliv. catechol protein nanoparticles TG-BSA NPs, and the scale bar is 100 nm. The results show that C, O, and N elements also exist in TG-BSA NPs, which is consistent with the elemental analysis results of TG-LP NPs.

### Example 3

600.00 mg of TG-HS solution (with a concentration of 10 mg/mL) was precisely weighed and added into 60.00 mL of ultrapure water, obtaining a mixed solution. The mixed solution was stirred to even. Then 10 mL of bovine serum albumin (BSA), lysozyme (LYZ), cytochrome C (CYC), β-lactoglobulin (bLG), pepsin, β-galactosidase (β-gal), hemoglobin (Hgb), fibrinogen (FGN), immunoglobulin G (IgG), horseradish peroxidase (HRP), and glucose oxidase (GOX) with concentrations of 1 mg/mL were added dropwise to the mixed solution, respectively, obtaining a mixture. The mixture was mechanically stirred (at a speed of 200 rpm for 2 h) and subjected to centrifugation (at a centrifugal force of 5,000 g for 10 min), collecting a precipitate. The precipitate was washed 3 times with water (at a centrifugal force of 5,000 g for 5 min), collecting a resulting precipitate, The resulting precipitate was labeled as TG-protein NPs, freeze-dried, and stored in a −20° C. refrigerator.

600.00 mg of TG-HS solution (with a concentration of 10 mg/mL) was precisely weighed and added into 60.00 mL of ultrapure water, obtaining a mixed solution. The mixed solution was stirred to even. Then 10 mL of bovine serum albumin (BSA), lysozyme (LYZ), cytochrome C (CYC), β-lactoglobulin (bLG), pepsin, β-galactosidase (β-gal), hemoglobin (Hgb), fibrinogen (FGN), immunoglobulin G (IgG), horseradish peroxidase (HRP), and glucose oxidase (GOX) with concentrations of 1 mg/mL were added dropwise to the mixed solution, respectively, obtaining a mixture. The mixture was stirred with heating reflux at 100° C. (at a speed of 200 rpm for 2 h) and subjected to centrifugation (at a centrifugal force of 5,000 g for 10 min), collecting a precipitate. The precipitate was washed 3 times with water (at a centrifugal force of 5,000 g for 5 min), collecting a resulting precipitate, The resulting precipitate was labeled as TG-protein NPs (A), freeze-dried, and stored in a −20° C. refrigerator.

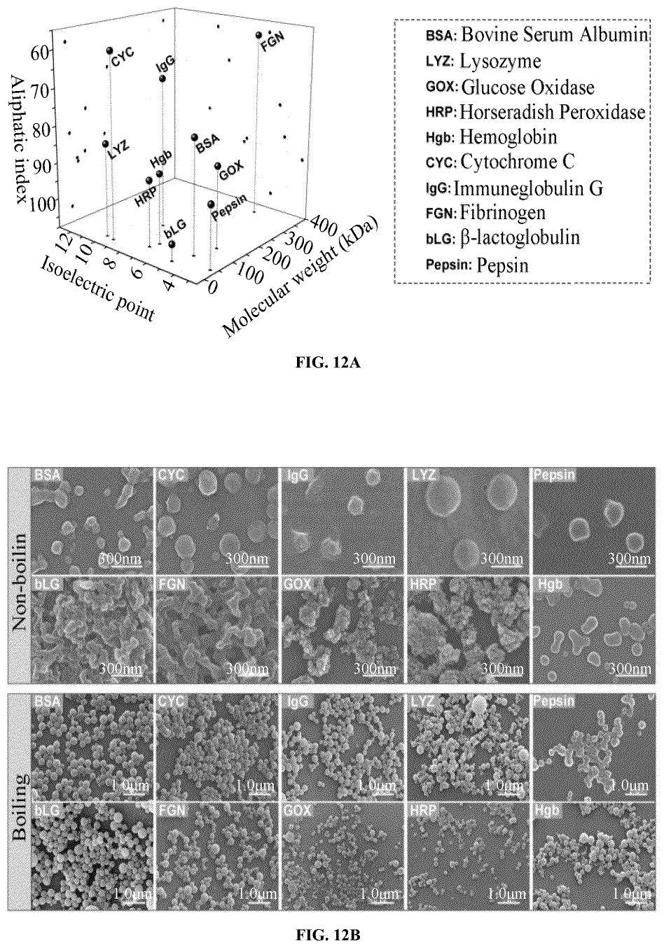

FIG. 12A is a model protein map showing different molecular weights, fat index, and isoelectric point; (x-axis: isoelectric point; y-axis: molecular weight; z-axis: aliphatic index); and b is an SEM change image showing different extraction states during the self-assembly of Quercus infectoria Oliv. extract TG-HS and 10 kinds of proteins to form nanoparticles.

As shown in FIG. 12A to FIG. 12B, compared with the rough surfaces of TG-LP NPs and TG-HP NPs, TG-protein NPs has a relatively smooth and complete surface. This is because the formation of TG-protein NPs involves a higher protein feeding amount, which contributes to the high adhesion between polyphenols and proteins. Moreover, it can be seen from the SEM results that spherical nanoparticles with more regular shapes can be obtained through self-assembly by heating reflux extraction. This result indicates that even if it is a different type of protein, Quercus infectoria Oliv. extract can form spherical nanoparticles with regular shapes with the proteins.

Protein Activity Verification:

The β-Gal assay in A549 cells was conducted according to the manufacturer's protocol of the β-Gal staining kit:

Principle: The β-Gal staining kit uses X-Gal as a substrate. Under the catalysis of senescence-specific β-Gal, a dark blue product can be generated, and cells expressing β-Gal that turn blue can thus be easily observed under a light microscope.

TG-β-gal NPs: No reflux heating was conducted during the preparation process, with simply mixing and stirring.

TG-β-gal NPs (Δ): The preparation process was accompanied by reflux stirring.

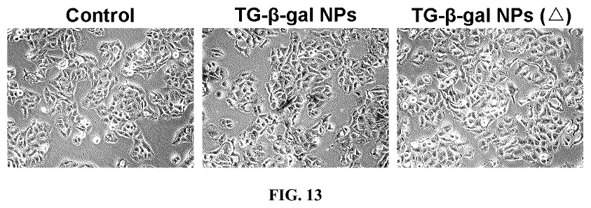

Methods: Taking β-gal as an example, catechol protein nanoparticles loaded with β-gal (TG-β-gal NPs) were co-incubated with A549 cells for 24 h, then washed with PBS 2 times, and fixated with a fixative for 10 min. The fixated cell was washed 3 times with PBS for 3 min each time, obtaining a washed cell. A working solution containing 5-bromo-4-chloro-3-indolyl-β-D-galactopyranoside (X-Gal) with a volume fraction of 5% was added to the washed cell, obtaining a mixture. The mixture was incubated overnight at 37° C. in a CO₂-free incubator, observed with an optical microscope, and photographed. The results are shown in FIG. 13.

As shown in FIG. 13, compared with the control group (Control) given free β-Gal, the cells in the TG-β-gal NPs group are blue, with a darker color; a part of the cells in the TG-β-gal NPs (Δ) group is blue, with a lighter color.

The results show that the catechol protein nanoparticles prepared by simple self-assembly can retain the activity of the protein. When the preparation process of the nanoparticles is accompanied by reflux heating, a part of the activity of the protein itself can be retained.

The above descriptions are merely preferred embodiments of the present disclosure. It should be noted that a person of ordinary skill in the art may further make several improvements and modifications without departing from the principle of the present disclosure, but such improvements and modifications should be deemed as falling within the protection scope of the present disclosure.

1. A method for preparing catechol nanoparticles, comprising:
adding a tannin compound-containing natural herb medicine into water to obtain a mixture, and subjecting the mixture to heating reflux extraction to obtain a herb medicine extract; and
subjecting the herb medicine extract to fractionation to obtain the catechol nanoparticles.

2. The method of claim 1, wherein the tannin compound-containing natural herb medicine comprises one drug selected from the group consisting of Quercus infectoria Oliv., Rhus chinensis Mill., and Sanguisorba officinalis L.;
a ratio of a mass of the tannin compound-containing natural herb medicine to a volume of water is 1 g: 8 mL; and
the heating reflux extraction is conducted by atmospheric reflux extraction or vacuum reflux extraction, wherein the atmospheric reflux extraction is conducted at 100° C. for 2 h, and the vacuum reflux extraction is conducted at 50° C. for 2 h.

3. The method of claim 1, wherein the fractionation is conducted by a process comprising the following steps: subjecting the herb medicine extract to first centrifugal separation to obtain a supernatant and the catechol nanoparticles; and the first centrifugal separation is conducted at a centrifugal force of 6,577 g for 10 min.

4. The method of claim 3, wherein the fractionation further comprises: subjecting the supernatant to second centrifugal separation to obtain the catechol nanoparticles; and the second centrifugal separation is conducted at a centrifugal force of 9,500 g for 10 min.

5. Catechol nanoparticles prepared by the method of claim 1, wherein the catechol nanoparticles are formed by self-assembly of a catechol and a protein, and have an average particle size of 413.89 nm±202.95 nm or 230.34 nm±59.48 nm.

6. A method for preparing catechol protein nanoparticles, comprising:
preparing a herb medicine extract by the method of claim 1; and
subjecting the herb medicine extract to first centrifugal separation to obtain a first supernatant, subjecting the first supernatant to second centrifugal separation to obtain a second supernatant, mixing the second supernatant, water, and a protein to obtain a mixture, subjecting the mixture to self-assembly under heating, and conducting separation to obtain the catechol protein nanoparticles.

7. The method of claim 6, wherein the protein comprises one protein selected from the group consisting of bovine serum albumin, lysozyme, cytochrome C, β-lactoglobulin, pepsin, β-galactosidase, hemoglobin, fibrinogen, immunoglobulin G, horseradish peroxidase, and glucose oxidase; and a mass ratio of the protein to the second supernatant is in a range of (10-300): 600.

8. The method of claim 6, wherein the heating is conducted at 100° C.; the self-assembly is conducted for 2 h; the separation is conducted by centrifugal separation; and the centrifugal separation is conducted at a centrifugal force of 5,000 g for 10 min.

9. Catechol protein nanoparticles prepared by the method of 6.

10. A drug carrier, comprising: the catechol nanoparticles of claim 5.

11. A drug carrier, comprising: the catechol protein nanoparticles of claim 9.

12. The method of claim 2, wherein the fractionation is conducted by a process comprising the following steps: subjecting the herb medicine extract to first centrifugal separation to obtain a supernatant and the catechol nanoparticles; and the first centrifugal separation is conducted at a centrifugal force of 6,577 g for 10 min.

13. The catechol nanoparticles of claim 5, wherein the tannin compound-containing natural herb medicine comprises one drug selected from the group consisting of Quercus infectoria Oliv., Rhus chinensis Mill., and Sanguisorba officinalis L.;
a ratio of a mass of the tannin compound-containing natural herb medicine to a volume of water is 1 gram: 8 mL; and
the heating reflux extraction is conducted by atmospheric reflux extraction or vacuum reflux extraction, wherein the atmospheric reflux extraction is conducted at 100° C. for 2 hours, and the vacuum reflux extraction is conducted at 50° C. for 2 hours.

14. The catechol nanoparticles of claim 5, wherein the fractionation is conducted by a process comprising the following steps: subjecting the herb medicine extract to first centrifugal separation to obtain a supernatant and the catechol nanoparticles; and the first centrifugal separation is conducted at a centrifugal force of 6,577 g for 10 min.

15. The catechol nanoparticles of claim 5, wherein the fractionation further comprises: subjecting the supernatant to second centrifugal separation to obtain the catechol nanoparticles; and the second centrifugal separation is conducted at a centrifugal force of 9,500 g for 10 min.

16. The catechol protein nanoparticles of claim 9, wherein the protein comprises one protein selected from the group consisting of bovine serum albumin, lysozyme, cytochrome C, β-lactoglobulin, pepsin, β-galactosidase, hemoglobin, fibrinogen, immunoglobulin G, horseradish peroxidase, and glucose oxidase; and a mass ratio of the protein to the second supernatant is in a range of (10-300): 600.

17. The catechol protein nanoparticles claim 9, wherein the heating is conducted at 100° C.; the self-assembly is conducted for 2 h; the separation is conducted by centrifugal separation; and the centrifugal separation is conducted at a centrifugal force of 5,000 g for 10 min.
